(19) United States
(12) Patent Application Publication
(10) Pub. No.: US 2011/0229437 A1
(43) Pub. Date: Sep. 22, 2011

(54) Method of Treating Asthma with Antiviral Agents

(75) Inventor: Richard H. Roberts, Lakewood, NJ (US)

(73) Assignee: MUTUAL PHARMACEUTICAL COMPANY, INC., Philadelphia, PA (US)

(21) Appl. No.: 12/884,780

(22) Filed: Sep. 17, 2010

Related U.S. Application Data
(60) Provisional application No. 61/243,449, filed on Sep. 17, 2009.

(51) Int. Cl. A61K 38/21; A61K 31/215; A61K 31/351; A61K 31/196; A61P 31/12

(52) U.S. Cl. 424/85.4; 514/529; 514/459; 514/563

(57) ABSTRACT

The present invention provides a method of treating asthma by administering a therapeutically effective amount of an antiviral agent to a patient. The antiviral agent may be a neuraminidase inhibitor, a viral fusion inhibitor, a protease inhibitor, a DNA polymerase inhibitor, a signal transduction inhibitor, a nucleoside reverse transcriptase inhibitor (NRTI), a non-nucleoside reverse transcriptase inhibitor (NNRTI) or an interferon. The antiviral agent may be administered to the patient by inhalation, nasally, intravenously, orally, subcutaneously, intramuscularly or transdermally. For example, the antiviral agent may be formulated for delivery as aerosols to the patient.

### CROSS REFERENCE TO RELATED APPLICATION

This application claims the benefit under 35 U.S.C. §119(e) of U.S. Provisional Application No. 61/243,449, filed on Sep. 17, 2009.

### FIELD OF THE INVENTION

The present invention relates to a pharmaceutical composition comprising an antiviral agent and methods of using this pharmaceutical composition to treat asthma.

### BACKGROUND OF THE INVENTION

Asthma is a chronic pulmonary disease primarily characterized by bronchoconstriction, airway inflammation, and excessive mucus production, all of which cause airway obstruction. Asthma symptoms include shortness of breath (dyspnea), wheezing, chest tightness, and cough, which can be life-threatening or even fatal. (The Merck Manual, 556-568 (17th ed., 1999).) Asthmatic patients also have increased airway sensitivity to a variety of stimuli, such as allergens, chemical irritants, atmospheric pollutants, respiratory infections, cold air and exercise. Thus, asthma is further characterized by acute episodes of additional airway narrowing via contraction of hyper-responsive bronchial smooth muscle.

The underlying causes of asthma are poorly understood. In most patients, the asthmatic attack consists of two phases. In the immediate phase, bronchoconstriction occurs due to spasms of the bronchial smooth muscle. The cells involved in the immediate phase include mast cells that release histamine, eosinophils, macrophages and platelets. Other factors secreted by the cells of the immune system include leukotrienes, prostaglandins, and platelet-activating factor which may be implicated in the pathology of asthma. The late phase is induced by inflammatory mediators released from activated, cytokine-releasing T cells and eosinophils, and is manifested by vasodilatation, edema, mucus secretion and bronchospasm.

Currently there is no cure for asthma. To suppress asthma symptoms and prevent attacks, asthma is managed pharmacologically through two types of treatments: long term control by use of anti-inflammatory agents and long-acting bronchodilators; and short term management of acute exacerbations through use of short-acting bronchodilators. Bronchodilators reverse bronchoconstriction by relaxing bronchial smooth muscle. Bronchodilators include the β₂-adrenoceptor agonists, the xanthines (e.g. theophylline) and the muscarinic-receptor antagonists (e.g. ipratropium bromide). The short-acting β₂-agonists, for example, salbutamol and terbutaline, are important for an immediate symptomatic relief, while long-acting β₂-agonists, such as salmeterol and formoterol are effective in the long term control of asthma and in the treatment of severe asthma.

Anti-inflammatory agents are capable of lessening or preventing inflammation in both asthma phases; such anti-inflammatory agents include corticosteroids (e.g., beclomethasone and budesonide), leukotriene antagonists, and histamine H1-receptor antagonists.

Most of the drugs available for the treatment of asthma are only effective in a limited number of patients. In addition, corticosteroids, the most commonly prescribed anti-inflammatory agents, can have considerable side effects, including oropharyngeal candidiasis, dysphonia, osteoporosis, mood disturbances, increased appetite and loss of glucose control in diabetics. (U.S. Pat. No. 7,553,487). β₂-agonists have been linked to deterioration in lung function and an increased risk of death. Moreover, the use of β₂-agonists is often associated with cardiovascular effects, such as altered pulse rate, blood pressure and electrocardiogram results. In rare cases, the use of β₂-agonists can produce hypersensitivity reactions, such as urticaria, angioedema, rash and oropharyngeal edema. Continuous treatment of asthmatic patients with the bronchodilator ipratropium bromide or fenoterol resulted in a faster decline in lung function, when compared with treatment based on need, indicating that they are not suitable for maintenance treatment. The most common immediate adverse effect of β₂-adrenergic agonists is tremors. β₂-adrenergic agonists at high doses may cause a fall in plasma potassium, dysrhythmias, and reduced arterial oxygen tension. (U.S. Pat. No. 7,405,207).

Development of a pharmaceutical composition that is able to effectively treat asthma, while reducing adverse side effects, is still needed.

We have unexpectedly discovered that antiviral agents are capable of alleviating asthma symptoms without co-administration of anti-inflammatory agents or bronchodilators. Alternatively, when administered in combination with anti-inflammatory agents or bronchodilators, the antiviral agents may allow lower required dosage of co-administered, anti-inflammatory agents or bronchodilators.

### SUMMARY OF THE INVENTION

It is an object of the present invention to provide a method of treating asthma in a patient comprising administering to the patient a therapeutically effective amount of an antiviral agent. The antiviral agent may be administered to the patient by inhalation, nasally, intravenously, orally, subcutaneously, intramuscularly or transdermally. For example, the antiviral agent may be formulated for delivery as aerosols to the patient.

The administration of the antiviral agent alleviates asthma symptoms. There may be at least about a 10% increase, a 20% increase, or a 30% increase, in forced expiratory volume in 1 second (FEV₁) or peak expiratory flow rate (PEFR) within about 30 minutes to about 14 days, within about 2 hours to about 12 days, within about 1 day to about 11 days, or within about 2 days to about 10 days after administration of the antiviral agent.

The antiviral agent may be a neuraminidase inhibitor, a viral fusion inhibitor, a protease inhibitor, a DNA polymerase inhibitor, a signal transduction inhibitor, a nucleoside reverse transcriptase inhibitor (NRTI), a non-nucleoside reverse transcriptase inhibitor (NNRTI) or an interferon.

The present invention further provides a pharmaceutical composition for treatment of asthma in a patient containing a therapeutically effective amount of an antiviral agent. The pharmaceutical composition may be formulated for delivery of the antiviral agent as aerosols to the patient. The pharmaceutical composition can be administered by inhalation, nasally, intravenously, orally, subcutaneously, intramuscularly or transdermally.

The present invention also provides an article of manufacture comprising a pharmaceutical formulation of an antiviral agent for treatment of asthma and printed matter indicating that the pharmaceutical formulation should be inhaled or swallowed by, or injected or otherwise administered into a patient suffering from asthma.

### DETAILED DESCRIPTION OF THE INVENTION

The present invention provides a pharmaceutical composition containing at least one antiviral agent and methods of using this pharmaceutical composition to treat asthma.

The present invention provides a method of treating asthma in a patient comprising administering to the patient a therapeutically effective amount of an antiviral agent. Also provided in the present invention is a pharmaceutical composition for treatment of asthma in a patient comprising a therapeutically effective amount of an antiviral agent. The antiviral agent is administered in a therapeutically effective amount to a patient suffering from asthma. The amount of the antiviral agent actually administered can be determined by a physician or other licensed healthcare professional, in light of the relevant circumstances, including the condition to be treated, the route of administration, the actual agent administered and its relative activity, the age, weight, and response of the individual patient, and the severity of the patient's symptoms. Suitable dosage of the antiviral agent for administration may range from about 0.1 μg/day to about 2 g/day, from about 1 μg/day to about 1 g/day, from about 5 μg/day to about 500 mg/day, from about 10 μg/day to about 300 mg/day, or from about 1 mg/day to about 200 mg/day.

The antiviral agent can be administered in a periodic dose, such as multiple doses per day, daily, multiple times per week, or weekly. The antiviral agent can also be administered based upon the patient's need. The treatment regimen may require administration over extended periods of time, for example, for several weeks or months; the treatment regimen may require administration over years and may require one or more repetitions as needed to alleviate asthma symptoms. The antiviral agent may be administered to the patient by inhalation, nasally, intravenously, orally, subcutaneously, intramuscularly or transdermally.

The antiviral agents are capable of alleviating asthma symptoms without co-administration of other therapeutic agents, such as anti-inflammatory agents or bronchodilators. Alternatively, the present antiviral agent can be administered in combination with one or more therapeutic agents, such as anti-inflammatory agents and β₂ adrenergic receptor agonists.

The antiviral agent may be any agent that inhibits entry of an infectious virus into a cell, or replication of an infectious virus in a cell. There are several stages within the process of viral infection which can be blocked or inhibited by antiviral agents. These stages include, attachment of the virus to the host cell (inhibited by, for example, suitable immunoglobulin or binding peptides), uncoating of the virus (inhibited by, e.g., amantadine), synthesis or translation of viral mRNA (inhibited by, e.g., interferon), replication of viral RNA or DNA (inhibited by, e.g., nucleoside analogues), maturation of new viral proteins (inhibited by, e.g., protease inhibitors), and budding and release of the virus (inhibited by, e.g., neuraminidase inhibitor).

Although antiviral agents fall within a general class of compounds or agents, the scope of the present invention is not limited to any specific physiological mechanism affected by the antiviral agents. For example, the antiviral agents may alleviate asthma symptoms through a defined antiviral mechanism, mediation of the immune system, inflammatory mechanism, or any other suitable physiological mechanism that enables the antiviral agents to alleviate asthma symptoms.

Useful antiviral agents include, but are not limited to, neuraminidase inhibitors, viral fusion inhibitors, protease inhibitors, DNA polymerase inhibitors, signal transduction inhibitors, reverse transcriptase inhibitors (such as nucleoside reverse transcriptase inhibitors and non-nucleoside reverse transcriptase inhibitors), interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors (e.g., CCR5 inhibitors/antagonists) and their functional analogs. Saxena et al., Future Virology, 4 (3): 209-215 (2009).

Neuraminidase inhibitors may include oseltamivir, zanamivir and peramivir. Viral fusion inhibitors may include cyclosporine, maraviroc, enfuviritide and docosanol. Protease inhibitors may include saquinavir, indinarvir, amprenavir, nelfinavir, ritonavir, tipranavir, atazanavir, darunavir, zanamivir and oseltamivir. DNA polymerase inhibitors may include idoxuridine, vidarabine, phosphonoacetic acid, trifluridine, acyclovir, foscarnet, ganciclovir, penciclovir, cidofovir, famciclovi, valaciclovir and valganciclovir. Signal transduction inhibitors include resveratrol and ribavirin. Nucleoside reverse transcriptase inhibitors (NRTIs) may include zidovudine (ZDV, AZT), lamivudine (3TC), stavudine (d4T), zalcitabine (ddC), didanosine (2′,3′-dideoxyinosine, ddI), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz. Viral entry inhibitors may include Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof.

Antiviral agents also include immunoglobulins (antibodies) used in immunoglobulin therapy for the prevention of viral infection. Immunoglobulin therapy functions by binding to extracellular virions and preventing them from attaching to and entering cells which are susceptible to the viral infection. In general there are two types of immunoglobulin therapies: normal immunoglobulin therapy and hyper immunoglobulin therapy. Normal immunoglobulin therapy utilizes antibodies which is prepared from the pooled serum of normal blood donors. This pooled product contains low titers of antibody to a wide range of human viruses. Hyper immunoglobulin therapy utilizes antibodies prepared from the serum of individuals who have high titers of specific antibodies to a particular virus. Examples of hyper-immune globulins include zoster immunoglobulin, human rabies immunoglobulin, hepatitis B immunoglobulin, and RSV immunoglobulin.

Antiviral agents may also include, but are not limited to, the following: acemannan; alovudine; alvircept sudotox; aranotin; arildone; atevirdine mesylate; avridine, carbovir, cipamfylline; clevadine, crixivan, cytarabine; desciclovir; dideoxyinosine, dideoxycytidine, disoxaril, edoxudine; enfuvirtide, entecavir, enviradene; enviroxime; famciclovir; famotine; fiacitabine; fialuridine; floxuridine, fosarilate; fosfonet, gancyclovir, kethoxal; levovirin, lobucavir; lopinovir, memotine, methisazone; moroxydine, pirodavir, pleconaril, podophyllotoxin, rimantadine, sequanavir, somantadine, sorivudine, stallimycine, statolon; tilorone; tromantadine, valacyclovir, viramidine, viroxime, xenazoic acid, zalcitabine; zerit, zinviroxime, pyridine, α-methyl-1-adamantanemethylamine, hydroxy-ethoxymethylguanine, adamantanamine, 5-iodo-2′-deoxyuridine, trifluorothymidine, adenine arabinoside, 2′,3′-dideoxynucleosides such as 2′,3′-didoxycytidine, 2′,3′-dideoxyadenosine, 2′,3′-didoxyinosine, 2′,3′-didehydrothymidine, co-trimoxazole, 9-[2-(R)-[[bis[Risopropoxy-carbonyl)oxy]-methoxy]phosphinoyl]methoxy]pro-pyl]adenine, (R)-9-[2-(phosphonomethoxy)-propyl]adenine, tenofivir disoproxil, TAT inhibitors such as 7-chloro-5-(2-pyrryl)-3H-1,4-benzodiazepin-2(H)-one or nucleic acids that comprise one or more unmethylated CpG sequences essentially as disclosed in, e.g., U.S. Pat. No. 6,194,388.

Viral respiratory infections exert considerable influence on airway function and asthma in all age groups. In infancy, respiratory viruses such as respiratory syncytial virus (RSV) cause episodes of wheezing that may be recurrent but are largely transient. In addition, early viral infections may be able to affect the development of the immune system and modify the subsequent risk of allergy and asthma. In children and adults with established asthma, respiratory viral infections by common cold viruses such as rhinovirus, frequently trigger asthma exacerbations. Folkerts et al., Virus-induced Airway Hyperresponsiveness and Asthma, Am. J. Respir. Crit. Care Med. 157: 1708-1720 (1998). U.S. Patent Publication No. 20080118516. In addition, infectious agents such as rhinovirus, RSV, human metapneumovirus (hMPV), influenza virus, adenovirus, parainfluenza virus, and coronavirus are frequently identified in respiratory secretions from children with asthma exacerbations. Murray et al., Study of modifiable risk factors for asthma exacerbations: virus infection and allergen exposure increase the risk of asthma hospital admissions in children. Thorax 61(5):376 (2006). Bronchial epithelial cells in asthmatic patients had significantly impaired interferon-β production when compared with healthy subjects. Wark, et al. Asthmatic bronchial epithelial cells have a deficient innate immune response to infection with rhinovirus. Journal of Experimental Medicine, 201 (6): 937-947 (2005).

The antiviral agents are capable of alleviating asthma symptoms without co-administration of other therapeutic agents, such as anti-inflammatory agents or bronchodilators. Alternatively, the present antiviral agent can be administered in combination with one or more therapeutic agents such as: anti-inflammatory agents (e.g., corticosteroids and non-steroidal anti-inflammatory agents (NSAIDs), β₂ adrenergic receptor agonists, antibiotics, antichlolinergic agents, and antihistamines. The present pharmaceutical composition may contain both the antiviral agent and the co-administered therapeutic agents. The antiviral agent and the co-administered therapeutic agents may also be in different pharmaceutical compositions.

As used herein, the term “therapeutically effective amount” is intended to include an amount of at least one antiviral agent of the present invention or an amount of the combination of agents including the antiviral agent, that is effective to treat or prevent asthma, or to treat the symptoms of asthma in a host. The term “treatment” or “treating” as used herein refers to alleviating the symptoms of asthma, arresting or relieving continued development of asthma. One indicator of the antiviral agent being therapeutically effective is improved forced expiratory volume in 1 second (FEV₁). Other indicators may include improved FEV₁/forced vital capacity (FVC) ratio, and peak expiratory flow rate (PEFR), as well as decreased airway resistance or decreased airway hyperresponsiveness. The measurement of pulmonary functions is described more fully below.

The co-administration of the antiviral agents with other therapeutic agents, such as anti-inflammatory agents or bronchodilators, may allow for lower dosage of the anti-inflammatory agents or bronchodilators. For example, the administration of an antiviral agent makes it possible to use lower dosage of short-acting β₂-agonists when managing asthma exacerbations, and/or lower dosage of long-acting β₂-agonists or corticosteroids for long-term treatment of asthma. The combination of compounds is preferably a synergistic combination. Synergy occurs when the effect of the agents when administered in combination is greater than the additive effect of the agents when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

To deliver the antiviral agent directly to the respiratory tract, the antiviral agent may be used to prepare pharmaceutical formulations for administration by inhalation. Typical delivery systems for inhalable agents include nebulizer inhalers, dry powder inhalers (DPI), and metered-dose inhalers (MDI).

Nebulizer devices produce a stream of high velocity air that causes a therapeutic agent in the form of liquid to spray as a mist which is carried into the patient's respiratory tract. The therapeutic agent is formulated in a liquid form such as a solution or a suspension of particles of respirable size. In one embodiment, the particles are micronized. The term “micronized” is defined as having about 90% or more of the particles with a diameter of less than about 10 μm. Suitable nebulizer devices are provided commercially, for example, by PARI GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The present antiviral agent can be formulated for use in a nebulizer device as an aqueous solution or as a liquid suspension.

The antiviral agents may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent or/and a lubricant nitrogen-containing polymer. U.S. Pat. Nos. 6,313,176 and 7,563,768.

DPI devices typically administer a therapeutic agent in the form of a free flowing powder that can be dispersed in a patient's air-stream during inspiration. DPI devices which use an external energy source may also be used in the present invention. In order to achieve a free flowing powder, the therapeutic agent can be formulated with a suitable excipient (e.g., lactose). A dry powder formulation can be made, for example, by combining dry lactose having a particle size between about 1 μm and 100 μm with micronized particles of the antiviral agent and dry blending. Alternatively, the antiviral agent can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl₃F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing respirable particles, and formulations and devices suitable for inhalation dosing see U.S. Pat. Nos. 6,268,533, 5,983,956, 5,874,063, and 6,221,398, and WO 99/53901, WO 00/61108, WO 99/55319 and WO 00/30614.

In instances where aerosol administration is appropriate, the antiviral agent can be formulated for delivery as aerosols using standard procedures. As used herein, the term “aerosol” refers to a suspension of fine solid particles or liquid solution droplets in a gas, which is capable of being inhaled into the respiratory tract. Specifically, aerosol includes a gas-borne suspension of droplets of an antiviral agent of the instant invention, as may be produced in an MDI or nebulizer, or in a mist sprayer. Aerosol also includes a dry powder composition of an antiviral agent of the instant invention suspended in air or other carrier gas, which may be delivered by an inhaler device. Ganderton & Jones, Drug Delivery to the Respiratory Tract, Ellis Horwood (1987). Gonda (1990) Critical Reviews in Therapeutic Drug Carrier Systems 6:273-313. Raeburn et al., (1992) Pharmacol. Toxicol. Methods 27:143-159.

The present pharmaceutical composition may be administered by any method known in the art, including, without limitation, oral, nasal, subcutaneous, intramuscular, intravenous, transdermal, rectal, sub-lingual, mucosal, ophthalmic, spinal, intrathecal, intra-articular, intra-arterial, sub-arachinoid, bronchial and lymphatic administration, and other dosage forms for systemic delivery of active ingredients. The composition may be delivered topically.

To prepare such pharmaceutical dosage forms, one or more of the antiviral agents are admixed with a pharmaceutical carrier according to conventional pharmaceutical compounding techniques. The carrier may take a wide variety of forms depending on the form of preparation desired for administration.

For example, when preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. If desired, tablets may be sugar coated or enteric coated by standard techniques.

For parenteral formulations, the carrier will usually comprise sterile water, although other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets (including orally dissolving tablets), lozenges, or other sublingual administration each containing a predetermined amount of the active ingredient as a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which usually is isotonic with the blood of the recipient (e.g., physiological saline solution). Such formulations may include suspending agents, thickening agents and liposomes or other microparticulate systems which are designed to target the compound to blood components or one or more organs. The formulations may be presented in unit-dose or multi-dose form.

Parenteral administration may comprise any suitable form of systemic delivery or delivery directly to the CNS. Administration may be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer. If the composition is in the form of a gel, the composition may be rubbed onto a membrane of the patient, for example, the skin, preferably intact, clean, and dry skin, of the shoulder or upper arm and or the upper torso, and maintained thereon for a period of time sufficient for delivery of the antiviral agent to the blood serum of the patient. The composition of the present invention in gel form may be contained in a tube, a sachet, or a metered pump. Such a tube or sachet may contain one unit dose of the composition. A metered pump may be capable of dispensing one metered dose of the composition.

Formulations of the invention may further include one or more accessory ingredient(s) selected from diluents, buffers, flavoring agents, binders, disintegrants, surface active agents, thickeners, lubricants, preservatives (including antioxidants), and the like.

The formulation of the present invention can have immediate release, sustained release, delayed-onset release or any other release profile known to one skilled in the art.

Dosage forms for the semisolid topical administration of the antiviral agents of this invention include ointments, pastes, creams, lotions, and gels. The dosage forms may be formulated with mucoadhesive polymers for sustained release of active ingredients at the area of application to the skin. The active compound may be mixed under sterile conditions with a pharmaceutically acceptable carrier, and with any preservatives, buffers, or propellants, which may be required. Such topical preparations can be prepared by combining the compound of interest with conventional pharmaceutical diluents and carriers commonly used in topical liquid, cream, and gel formulations.

Ointment and creams may, for example, be formulated with an aqueous or oily base with the addition of suitable thickening and/or gelling agents. Such bases may include water and/or an oil including, but not limited to, liquid paraffin or a vegetable oil including, but not limited to, peanut oil or castor oil. Thickening agents which may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and, in general, also include one or more of the following: stabilizing agents, emulsifying agents, dispersing agents, suspending agents, thickening agents, coloring agents, perfumes, and the like. The ointments, pastes, creams and gels also may contain excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Suitable excipients, depending on the hormone, include petrolatum, lanolin, methylcellulose, sodium carboxymethylcellulose, hydroxpropylcellulose, sodium alginate, carbomers, glycerin, glycols, oils, glycerol, benzoates, parabens and surfactants. It will be apparent to those of skill in the art that the solubility of a particular compound will, in part, determine how the compound is formulated. An aqueous gel formulation is suitable for water soluble compounds. Where a compound is insoluble in water at the concentrations required for activity, a cream or ointment preparation will typically be preferable. In this case, oil phase, aqueous/organic phase and surfactant may be required to prepare the formulations. Thus, based on the solubility and excipient-active interaction information, the dosage forms can be designed and excipients can be chosen to formulate the prototype preparations.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients including, but not limited to, antimicrobial agents, particularly antibiotics, anesthetics, analgesics, and antipruritic agents.

If a topical composition or one that involves administration to a mucosal surface such as the nasal or rectal epithelium is desired, the composition of the present invention may contain an enhancer, that is, a compound capable of increasing the rate of passage of an antiviral agent through a body membrane. He et al. Mechanistic Study of Chemical Skin Permeation Enhancers with Different Polar and Lipophilic Groups. Pharmaceutical Sciences 93(6): 1415 -1430 (2004).

In another embodiment, the present pharmaceutical composition is in the form of an intranasal spray. The intranasal spray may comprise an antiviral agent, a liquid carrier, and suitable surfactants. Any suitable surfactant or mixture of surfactants can be used in the practice of the present invention, including, for example, anionic, cationic, and non-ionic surfactants.

The composition may comprise a carrier that is capable of solubilizing one or more of the ingredients comprising the composition of the present invention. Essentially any suitable carrier or mixture of carriers that is a suitable vehicle for the composition of the present invention can be used in the practice thereof. Preferred carriers are characterized by at least one of the following properties: low irritability or no irritability to the target membrane; being listed in the National Formulary or the U.S. Pharmacopeia; capability to enhance penetration of the antiviral agent across a membrane; and capability to perform an additional function in the composition, for example, function also as an emollient, a humectant, a plasticizer, a lubricating agent, and/or a protein stabilizer. The carrier is present in the composition in a concentration effective to serve as a suitable vehicle for the compositions of the present invention. For guideline purposes, it is believed that most applications will involve the use of the carrier in an amount of about 40% (w/w) to about 98% (w/w) of the total composition; other ranges include from 50% (w/w) to about 80% (w/w).

In addition to the particles in accordance with this embodiment of the present invention, the dosage forms may also include glidants, lubricants, binders, sweeteners, flavoring and coloring components. Any conventional sweetener or flavoring component may be used. Combinations of sweeteners, flavoring components, or sweeteners and flavoring components may likewise be used.

Examples of binders which can be used include acacia, tragacanth, gelatin, starch, cellulose materials such as methyl cellulose and sodium carboxy methyl cellulose, alginic acids and salts thereof, magnesium, aluminium silicate, polyethylene glycol, guar gum, polysaccharide acids, bentonites, sugars, invert sugars and the like. Binders may be used in an amount of up to 60% (w/w) or about 10% (w/w) to about 40% (w/w) of the total composition.

Coloring agents may include titanium dioxide, and dyes suitable for food such as those known as F.D. & C. dyes and natural coloring agents such as grape skin extract, beet red powder, beta-carotene, annatto, carmine, turmeric, paprika, etc. The amount of coloring used may range from about 0.1% (w/w) to about 3.5% (w/w) of the total composition.

Flavors incorporated in the composition may be chosen from synthetic flavors oils and flavoring aromatics and/or natural oils, extracts from plants, leaves, flowers, fruits and so forth and combinations thereof. These may include cinnamon oil, oil of wintergreen, peppermint oils, clove oil, bay oil anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors may be present in an amount ranging from about 0.05% (w/w) to about 3% (w/w) based on the total composition.

The composition of the present invention may comprise a preservative capable of preventing oxidation of the components of the composition, microbial growth, or contamination. Essentially any suitable preservative or mixture of preservatives may be used in the practice of the present invention. Preferred preservatives include: food additive anti-microbial agents, for example, quaternary ammonium salts, sorbic acid, acetic acid, and benzoic acid or salts thereof; and antioxidants, for example, Vitamin C, Vitamin E, butylated hydroxyanisole (BHA), and butylated hydroxytoluene (BHT). Examples of preferred antimicrobial preservatives include benzalkonium chloride and cetyl pyridinium chloride. The preservative is present in the composition in a concentration effective to inhibit microbial growth, the oxidation of the components of the composition, or contamination of the composition. Effective percentages range from about 0.0001% (w/w) to about 1.0% (w/w) or 0.005% (w/w) to about 0.1% (w/w) of the total composition.

Compositions which use a thickening agent may require neutralization to achieve a desired thickening effect for the composition. For example, carbomers, being acidic molecules, require neutralization, preferably to a pH of between 3 and 9, to achieve their maximum viscosity. Essentially any suitable neutralizing agent or mixture of neutralizing agents can be used. Preferred neutralizing agents are characterized by at least one of the following properties: a pKa greater than about 9, with a pKa greater than about 9.5 being particularly preferred; and being compendial, i.e., being approved for use by governmental agencies in pharmaceutical formulations. Examples of the neutralizing agents that exhibit both of the above properties include triethanolamine, tromethamine, tris amino, triethylamine, 2-amino-2-methyl-1-propanol, sodium hydroxide, ammonium hydroxide, and potassium hydroxide. The choice of particular neutralizing agent for use in the present application should take into account the thickening agent or solvent being used. Noveon publication TRS-237. The pH of the composition may range from about 3 to about 9, about 4 to about 8 or about 5 to about 7.

The composition of the present invention may include additional ingredients which are art-recognized and in art-recognized quantities. For example, materials may be added to modify the rheology, feel, slip, stability, humectancy, fragrance and other desirable physical properties that a practitioner may deem desirable. In addition, buffers may be added to maintain the composition at a certain pH.

In situations where a unit dose is applied, one or more of such unit doses may be administered to a patient, depending upon the condition to be treated, and the frequency of administration. The number of such unit doses may be increased or decreased as needed, depending upon, for example, whether a desired clinical effect has been achieved.

The composition of the present invention may be formulated by the use of conventional means, for example, by mixing and stiffing the ingredients. Conventional equipment may be used.

The present invention provides an article of manufacture containing a pharmaceutical formulation having at least one antiviral agent, and printed matter indicating that the pharmaceutical formulation is used for treatment, prevention or amelioration of one or more symptoms of asthma. In one embodiment, printed matter indicates that the pharmaceutical formulation should be inhaled by a patient suffering from asthma. The article of manufacture may contain packaging material. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. See, e.g., U.S. Pat. Nos. 5,323,907, 5,052,558 and 5,033,252 for additional examples of pharmaceutical packaging materials. In one embodiment herein, the compositions are packaged with a nebulizer for direct administration of the composition to a subject.

Limitation of airflow in asthma is due mainly to bronchoconstriction, but airway edema, vascular congestion, and luminal occlusion with exudate may also contribute to asthma symptoms. This results in a reduction in forced expiratory volume in 1 second (FEV₁), FEV₁/forced vital capacity (FVC) ratio, and peak expiratory flow rate (PEFR), as well as an increase in airway resistance. Early closure of peripheral airway results in lung hyperinflation (air trapping), and increased residual volume, particularly during acute exacerbations. In more severe asthma, reduced ventilation and increased pulmonary blood flow result in mismatching of ventilation and perfusion, and bronchial hyperemia. Asthma may be accompanied by hypoxemia. Arterial Pa_{CO2} tends to be low due to increased ventilation. Airway hyperresponsiveness (AHR) is also the characteristic physiologic abnormality of asthma, and refers to the excessive bronchoconstrictor response to multiple inhaled triggers that would have no effect on normal airways. Chapters 245-248, Harrison's Principles of Internal Medicine, 17^{th} edition, published by McGraw-Hill Companies, Inc. 2008.

Accordingly, it is essential to monitor pulmonary functions in the management of asthma. This invention further includes a method of assessing the pulmonary condition of an asthmatic subject before, during and after treatment with an antiviral agent. Pulmonary function tests may be performed to obtain at least one pulmonary function value. The pulmonary function value is compared to a corresponding predetermined pulmonary function value. The pulmonary function values of the asthmatic subject before, during and after antiviral treatment can be compared.

Pulmonary function tests may measure (1) ventilatory function, (2) pulmonary circulation, or (3) gas exchange. Chapters 245-248, Harrison's Principles of Internal Medicine, 17^{th} edition, published by McGraw-Hill Companies, Inc. 2008.

Measurements of ventilatory function consist of quantification of the gas volume contained in the lungs under certain circumstances and the rate at which gas can be expelled from the lungs. The two measurements of lung volume commonly used for respiratory diagnosis are (1) total lung capacity (TLC); and (2) residual volume (RV). The volume of gas that is exhaled from the lungs in going from TLC to RV is the vital capacity (VC). Common clinical measurements of airflow are obtained from maneuvers in which the subject inspires to TLC and then forcibly exhales to RV. Three measurements are commonly made from a recording of forced exhaled volume versus time, i.e., a spirogram: (1) forced expiratory volume (FEV) in 1 second, or FEV₁, (2) forced vital capacity (FVC), and (3) forced expiratory flow (FEF) between 25 and 75% of the VC, or FEF_{25-75%}, also called the maximal midexpiratory flow rate (MMFR). Ventilatory function tests may also measure peak expiratory flow rate (PEFR), airway resistance (AR), and forced midexpiratory flow. PEFR may be measured by a peak flow meter.

Ventilatory function is measured under static conditions for determination of lung volumes and under dynamic conditions for determination of FEF. VC, expiratory reserve volume (ERV), and inspiratory capacity (IC) are measured by having the patient breathe into and out of a spirometer, a device capable of measuring expired or inspired gas volume while plotting volume as a function of time. Two techniques are commonly used to measure other volumes, such as RV, FRC, and TLC: helium dilution and body plethysmography. In the helium dilution method, the subject repeatedly breathes in and out from a reservoir with a known volume of gas containing a trace amount of helium. The helium is diluted by the gas previously present in the lungs and very little is absorbed into the pulmonary circulation. From knowledge of the reservoir volume and the initial and final helium concentrations, the volume of gas present in the lungs can be calculated. The helium dilution method may underestimate the volume of gas in the lungs if there are slowly communicating airspaces, such as bullae. In this situation, lung volumes may be measured more accurately with a body plethysmograph, a sealed box in which the patient sits while panting against a closed mouthpiece. Because there is no airflow into or out of the plethysmograph, the pressure changes in the thorax during panting cause compression and rarefaction of gas in the lungs and simultaneous rarefaction and compression of gas in the plethysmograph. By measuring the pressure changes in the plethysmograph and at the mouthpiece, the volume of gas in the thorax can be calculated using Boyle's law. Lung volumes and measurements made during forced expiration are interpreted by comparing the values measured with the values expected given the age, height, sex, and race of the patient. Chapters 245-248, Harrison's Principles of Internal Medicine, 17^{th} edition, published by McGraw-Hill Companies, Inc. 2008.

In some pulmonary function tests, the test is performed with the patient breathing mixture of gases with varying densities. Examples of such gases include air, helium, oxygen, nitrogen, carbon monoxide, carbon dioxide, and inert gases.

Gas exchange can be measured to evaluate the pulmonary function. The most commonly used measures of gas exchange are the partial pressures of O₂ and CO₂ in arterial blood, i.e., Pa_{O2} and Pa_{CO2}, respectively. These partial pressures do not measure directly the quantity of O₂ and CO₂ in blood but rather the driving pressure for the gas in blood.

Most commonly, P_{O2} is the measurement used to assess the effect of asthma on the oxygenation of arterial blood. Direct measurement of O₂ saturation in arterial blood by oximetry is also important in selected clinical conditions. A useful calculation in the assessment of oxygenation is the alveolar-arterial O₂ difference (PA_{O2}-Pa_{O2}), commonly called the alveolar-arterial O₂ gradient (or A—a gradient). This calculation takes into account the fact that alveolar and, hence, arterial P_{O2} can be expected to change depending on the level of alveolar ventilation, reflected by the arterial P_{CO2}. When a patient hyperventilates and has a low P_{CO2} in arterial blood and alveolar gas, alveolar and arterial P_{O2} will rise; conversely, hypoventilation and a high P_{CO2} are accompanied by a decrease in alveolar and arterial P_{O2}. These changes in arterial P_{O2} are independent of abnormalities in O₂ transfer at the alveolar-capillary level and reflect only the dependence of alveolar P_{O2} on the level of alveolar ventilation.

In order to determine the alveolar-arterial O₂ difference, the alveolar P_{O2} (PA_{O2}) must first be calculated. The equation most commonly used for this purpose, a simplified form of the alveolar gas equation, is

PA_{O2}=FI_{O2}×(P_{B}−P_{H2O})−PA_{CO2}/R

where FI_{O2}=fractional concentration of inspired O₂ (0.21 when breathing room air); P_{B}=barometric pressure (˜760 mmHg at sea level); P_{H2O}=water vapor pressure (47 mmHg when air is fully saturated at 37° C.); and R=respiratory quotient (the ratio of CO₂ production to O₂ consumption, usually assumed to be 0.8). If the preceding values are substituted into the equation for the patient breathing air at sea level, the equation becomes

PA_{O2}=150−1.25×Pa_{CO2}

The alveolar-arterial O₂ difference can then be calculated by subtracting measured Pa_{O2} from calculated PA_{O2}. The adequacy of CO₂ elimination is measured by the partial pressure of CO₂ in arterial blood, i.e., Pa_{CO2}.

Pulse oximetry is an alternative method for assessing oxygenation. Using a probe usually clipped over a patient's finger, the pulse oximeter calculates oxygen saturation (rather than Pa_{O2}) based on measurements of absorption of two wavelengths of light by hemoglobin in pulsatile, cutaneous arterial blood. Because of differential absorption of the two wavelengths of light by oxygenated and nonoxygenated hemoglobin, the percentage of hemoglobin that is saturated with oxygen, i.e., the Sa_{O2}, can be calculated and displayed instantaneously.

The increased AHR is normally measured by methacholine or histamine challenge with calculation of the provocative concentration that reduces FEV₁ by 20% (PC₂₀). This is rarely useful in clinical practice, but can be used in the differential diagnosis of chronic cough and when the diagnosis is in doubt in the setting of normal pulmonary function tests. Occasionally exercise testing is done to demonstrate the post-exercise bronchoconstriction if there is a predominant history of EIA. Allergen challenge may also be undertaken.

Imaging techniques such as chest roentgenography, computed tomography (CT), and Magnetic Resonance Imaging (MRI) may be used to evaluate the effectiveness of antiviral agents in treating asthma.

Pulmonary function tests also include challenge and exercise testing; measurements of respiratory muscle strength; and fibro-optic airway examination.

The administration of the present antiviral agents improves asthma symptoms. In certain embodiments of the present invention, there is at least about a 10% increase, about a 20% increase, or about a 30% increase in FEV₁ or PEFR within about 30 minutes to about 14 days, within about 2 hours to about 12 days, within about 1 day to about 11 days, or within about 2 days to about 10 days after administration of the antiviral agent. There may be at least about a 10% increase, about a 20% increase, or about a 30% increase in FVC, FEV₁/FVC ratio, FEF_{25-75%} (or MMFR) or forced midexpiratory flow within about 30 minutes to about 14 days, within about 2 hours to about 12 days, within about 1 day to about 11 days, or within about 2 days to about 10 days after administration of the antiviral agent. There may be at least about a 10% decrease, about a 20% decrease, or about a 30% decrease in airway resistance (AR) or airway hyperresponsiveness within about 30 minutes to about 14 days, within about 2 hours to about 12 days, within about 1 day to about 11 days, or within about 2 days to about 10 days after administration of the antiviral agent. In some embodiments, the increase in FEV1 or PEFR may range from about 10% to about 4 fold, from about 20% to about 3 fold, or from about 30% to about 2 fold within about 30 minutes to about 14 days, within about 2 hours to about 12 days, within about 1 day to about 11 days, or within about 2 days to about 10 days.

Procedures for studying the properties of mucus include rheology (for example, using a magnetic microrheometer); adhesivity to characterize the forces of attraction between an adherent surface and an adhesive system by measuring the contact angle between a mucus drop and a surface. Mucus transport by cilia can be studied through direct measurement, i.e., in situ mucus clearance. Transepithelial potential difference, which demonstrates the activity of the ion-transport system of the pulmonary epithelium, can be measured using appropriate microelectrodes. Quantitative morphology methods may be used to characterize epithelial surface condition. U.S. Pat. No. 7,531,500.

Assessment of health-related quality of life (QoL) is an important aspect of asthma management in clinical practice. Asthma-related QoL can be assessed by questionnaires including, but not limited to, St. George's Respiratory Questionnaire (SGRQ), Asthma Quality of Life Questionnaire (AQLQ), Living with Asthma Questionnaire (LWAQ), Asthma Control Questionnaire (ACQ), Mini Asthma Quality of Life Questionnaire (MiniAQLQ), Quality of Life for Respiratory Illness Questionnaire (QOLRIQ), Knowledge, Attitude, and Self-Efficacy Asthma Questionnaire (KASE-AQ), Asthma Symptom Utility Index (ASUI), University of Alabama at Birmingham (UAB) Functional Impairment Scale, Perceived Control of Asthma Questionnaire (PCAQ), Asthma Short Form or Rhinasthma. AAAAI Quality of Life Resources. Asthma—Adult/General. [online], [retrieved on Jul. 29, 2009]. Retrieved from the Internet<URL: http://www.aaaai.org/professionals/quality_of_life/asthma_adult.stm>

The present pharmaceutical composition may be used to treat other respiratory diseases, such as airway inflammation, allergy, impeded respiration, cystic fibrosis (CF), chronic obstructive pulmonary diseases (COPD), allergic rhinitis (AR), acute respiratory distress syndrome (ARDS), pulmonary hypertension, lung inflammation, bronchitis, airway obstruction, or bronchoconstriction.

The patient to be treated can be a primate, such as a human, or any other animal exhibiting asthma symptoms. While the method of the invention is especially adapted for the treatment of a human patient, it will be understood that the invention is also applicable to veterinary practice.

The following example is offered to illustrate, but not to limit, the claimed invention.

### EXAMPLE 1

An adult male with history of asthma had been taking a combination of inhaled steroid and β₂-agonist for many years with mixed results. He had been using Advair® two inhalations a day or Symbicort® four puffs a day. Advair® is a combination formulation containing fluticasone propionate (a corticosteroid) and salmeterol xinafoate (a long-acting β₂-adrenergic receptor agonist). Symbicort® contains two active ingredients which are delivered via a single inhaler: budesonide, a corticosteroid, and formoterol, a long-acting β₂-agonist. His peak flows were 600 to 650 liters per minute at best, but would deteriorate to levels around 250 to 350 liters per minute during exacerbations and often reached below 550 liters per minute without an attack. Over the years, he needed oral prednisone (a corticosteroid) treatment occasionally. Exercise tolerance was moderate to poor.

He exhibited flu like symptoms, and started taking Tamiflu® 75 mg. by mouth twice a day for 5 days. Within 12 hours, he began feeling better and myalgias were relieved; fever dropped from 39 C.° to 38 C.° within 1.5 days; the fatigue resolved within 1.5 days. Surprisingly, his asthma symptoms diminished as well. He breathed better than on 60 mg of prednisone per day. One week after completing his 5 day course of Tamiflu®, he ceased using the steroid/β₂-agonist inhaler due to the near absence of asthmatic symptoms. Five weeks after using Tamiflu®, he still did not need any steroid/β₂-agonist inhaler and his peak flows were 625 to 650 liters per minute. The level of relief was reported, by the patient, to be similar to the level of asthmatic relief that he experienced while taking prednisone 60 mg. per day. However, there had not been a complete disappearance of asthma symptoms, since exercise still caused some mild breathing tightness. He continued to use Singulair® since it had no side effects in him and he had been using Singulair® for years prior to this experience with Tamiflu®. Singulair® contains montelukast, a leukotriene receptor antagonist (LTRA) used for the maintenance treatment of asthma and to relieve symptoms of seasonal allergies.

### EXAMPLE 2

### Randomized, Double-blind, Placebo-Controlled, Assessment of Oseltamivir phosphate (Tamiflu®) in Patients with Not-Well-Controlled Asthma

Current asthma guidelines emphasize control of asthma. One component of control is the current burden, indicated by level of symptoms, rescue medication use, night time awakenings, and interference with normal activities. Patients not meeting the specified goals are considered to have “Not Well Controlled Asthma”. The guidelines further recommend steps of thereapy which are to used to achieve control. For many patients these therapeutic measures are sufficient to attained asthma control, but for at least 30%, even optimal doses of inhaled corticosteroids and long-acting beta-agonists continued for a year failed to achieve control in the GOAL study (Bateman et al. Can guideline defined asthma control be achieved? The Gaining Optimal Asthma Control study. Am J Respir Crit Care Med 2004;170:836-44). Therefore, identification of additional therapeutic options for the treatment of asthma are needed.

Oseltamivir is a neuraminidase inhibitor approved for the prophylaxis and treatment of infection by all strains of influenza viruses. A. Moscona. The neuraminidase inhibitors for influenza. N. Engl. J. Med. 2005;353:1363-73. As such, it is specific for the influenza virus and no other actions are listed. It is the purpose of this exploratory study to investigate the possible beneficial effects of oseltamivir in patients with asthma not adequately controlled on inhaled corticosteroids.

### Study Design:

This is a single-center study which will recruit 21 subjects 18-65 years of age with an established diagnosis of asthma who are currently on treatment with low to medium doses of inhaled corticosteroids and are not achieving asthma control as defined by the NHLBI Guidelines for the Diagnosis and Management of Asthma (2007). After meeting entry criteria, subjects will be randomized in a 2:1 ratio (14/7) to receive the recommended and approved 5-day course of oseltamivir phosphate or matching placebo. Assessments will be performed prior to randomization, after the first dose, after 2 weeks and after 4 weeks. The primary outcome will be the difference in the change in the Asthma Control Questionnaire score between the two groups.

### Subjects:

The subjects will be males or females 18 to 65 years of age with at least a two-year history of persistent asthma with documented reversibility within the last 12 months and a stable inhaled corticosteroid dose for at least one month.

Initial inclusion criteria include: male or female, 18-65 years of age; history of perennial physician-diagnosed asthma for at least two years; woman of reproductive potential using adequate birth control measures (no birth control measures are required of males, since oseltamivir is an FDA approved product); no abnormalities on physical examination, chest x-ray or laboratory studies which would place the patient at risk by participation or interfere with the interpretation of study results; on inhaled corticosteroids (fluticasone propionate 88-440 micrograms/day or equivalent) with no dose change in the previous 4 weeks; no tobacco use in the last 6 months and not over 10 pack-years total use; and FEV₁ between 50 and 80% of predicted with at least 12% improvement with inhaled albuterol at screening or historically within the previous 12 months.

Additional inclusion criteria for randomization is Asthma Control Questionnaire (ACQ) Score of 1.5 or greater.

Exclusion criteria include: asthma exacerbation requiring systemic corticosteroids, emergency room visit or hospitalization with 2 months of screening; history of intubation for asthma except in childhood (before age 12 years); history of COPD or any pulmonary disease that could interfere with interpretation of study results; Body Mass Index of 30 or more; history of untreated obstructive sleep apnea; any uncontrolled systemic disease which would place the subject at risk by participating or which would interfere with the interpretation of study result; a history of a respiratory infection within the 4 weeks prior to screening; use of an experimental drug within 30 days prior to screening; treatment with omalizumab within 3 months of screening; known positive test for hepatitis C or HIV; active drug or alcohol abuse; and planned elective surgery during the period of participation.

### Visits and Procedures:

Screening visits (1+1a and 1b as required):
1. Review and sign IRB approved Patient Consent Form; review inclusion and exclusion criteria.
2. Obtain screening laboratory safety studies: CBC, Biochemical panel, urinalysis and ECG.
3. Perform spirometry with albuterol reversibility (4 puffs).
4. Chest x-ray will be performed if not available from within the previous year.

If the subject meets medication and spirometric criteria as listed in the inclusion criteria, they will be administered the Asthma Control Questionnaire assessing symptoms over the previous 7 days. If they meet the additional inclusion criteria of a score of ≧1.5 they will be scheduled to return for the randomization visit within one week.

Further Screening Visits:
1. Subjects who are taking disallowed medications (long-acting beta-agonists, leukotriene pathway modifying agents, theophylline) will discontinue these medications and will return in 2 weeks to complete an ACQ covering the previous 7 days.
2. Subjects who score lower than 1.5 on the ACQ, with their consent, may have their inhaled corticosteroid dose reduced by 50% (for those with an ACQ score of 0-0.75 and by 25% for those with an ACQ score of 1.0-1.25). They will be scheduled to return in 4 weeks to complete an ACQ covering the previous 7 days. During this 4 week period they will be instructed to monitor symptoms, beta-agonist use and peak flows and will be provided with alert levels for each, based on their levels before corticosteroid reduction, which will trigger contact with the Clinical Research Center.
3. Any subject meeting the ACQ criteria at visit 1a, or 1b, will be scheduled for the randomization visit within 7 days.

### Pre-Randomization Visits:

Once subjects have qualified for randomization (meeting all inclusion criteria including an ACQ score of 1.5 or greater) they will be instructed in and commence twice daily peak flow monitoring and recording of symptoms and short-acting beta-agonist use. The following studies will be scheduled and performed prior to randomization (Visit 1c):
1. Mannitol inhalation challenge (mannitol will be used if it has been approved by the FDA by the time of study initiation).
2. Exhaled nitric oxide.
3. Serum ECP.
4. Asthma Quality of Life Questionnaire.

### Randomized Visit:

1. Review of inclusion and exclusion criteria including laboratory studies and chest x-ray if performed.
2. Review of PEF, symptom and beta-agonist use diaries for completeness.
3. Review of adverse events.
4. Complete ACQ covering the previous 7 days.
5. Baseline spirometry.
6. Baseline vital signs.
7. Rapid pregnancy test for women of childbearing potential.
8. Administer blinded study medication (twenty-one subjects will be randomized in a 2:1 ratio to receive oseltamivir 75 mg twice daily or matching placebo twice daily for 5 days).
9. Following dosing at 6:00 AM they will have repeat spirometry after 1, 2, 3, 6, 9 and 12 hours with second dosing at 12 hours.
10. After the second dosing they will be discharged from the unit, provided with 8 capsules of blinded study medication with instructions to take one capsule twice daily at approximately 12 hour intervals, to continue recording symptoms, peak flows, adverse events and beta-agonist use twice daily and to return in 2 weeks to the Clinical Research Unit. If, during the next 4 weeks, any subject experiences symptoms consistent with a respiratory tract infection (sore throat, fever, profuse rhinorrhea) they will have a rapid screening test for influenza and if positive they will be discontinued from the study.

### 2-Week Visit:

Subjects will return at approximately 7:00 AM two weeks following randomization for the following activities:
1. Review of diaries for symptoms, adverse events, beta-agonist use and peak flow results.
2. ACQ covering the previous 7 days.
3. Spirometry.
4. Exhaled nitric oxide.
5. Serum ECP.

### 4-Week Visit:

Subjects will return at approximately 7:00 AM for the following activities:
1. Review of diaries for symptoms, adverse events, beta-agonist use and peak flow results.
2. ACQ covering the previous week. 3. Spirometry
4. Exhaled nitric oxide
5. Serum ECP
6. AQLQ
7. Mannitol inhalation challenge
   Table 1 lists the various activities/studies for the scheduled visits.

TABLE 1

| | Visit | Visit | Visit | Visit | Randomi- | 2 | 4 |
|---|---|---|---|---|---|---|---|
| Activity | 1 | 1a | 1b | 1c | zation | Week | Week |
| Sign Consent | X | | | | | | |
| Review In | X | | | | X | | |
| and Ex | | | | | | | |
| Screening | X | | | | | | |
| Labs | | | | | | | |
| ECG | X | | | | | | |
| Spirometry | X | | | | Pre- | Pre- | Pre- |
| | | | | | only | only | only |
| Pre- & Post- | | | | | | | |
| Chest X-ray | (X) | | | | | | |
| Asthma | (X) | (X) | (X) | | X | X | X |
| Control | | | | | | | |
| Questionnaire | | | | | | | |
| Instruction in | X | | | | | | |
| PEF & Diary | | | | | | | |
| Review Diary | | (X) | (X) | (X) | X | X | X |
| & Adverse | | | | | | | |
| Events | | | | | | | |
| Mannitol | | | | X | | | X |
| Challenge | | | | | | | |
| FeNO | | | | X | | X | X |
| Serum ECP | | | | X | | X | X |
| AQLQ | | | | X | | | X |
| Vital Signs | | | | | X | | |
| Pregnancy | | | | | (X) | | |
| Test | | | | | | | |
| Administer | | | | | Twice 12 | | |
| Test Drug | | | | | hours apart | | |
| 12-hour Serial | | | | | X | | |
| Spirometry | | | | | | | |

Note: ( ) means the activity will be done as indicated.

### Statistical Analysis:

The data will be analyzed comparing change from baseline within the active treatment group and between the two groups by analysis of covariance (ANCOVA) or other appropriate statistical methods. The primary outcome is the change in ACQ score from baseline (randomization) to week 4. Additional outcomes will be: Acute change in FEV1 following first dose, change in FEV1 at 2 and 4 weeks, mean change in peak expiratory flow for each of the four weeks, change in exhaled nitric oxide and serum eosinophil cationic protein levels at 2 and 4 weeks, change in Asthma Quality of Life Questionnaire from baseline to 4 weeks, change in symptoms and beta-agonist use averaged weekly over the 4 weeks, and change in mannitol or methacholine PC20 over 4 weeks.

### Study Methods:

### Mannitol Challenge

The mannitol test will be carried out as per the standard laboratory protocol using the commercially available mannitol test kit (known as Aridol™ or Osmohale™ Pharmaxis Ltd, AUS). Brannan et The safety and efficacy of inhaled dry powder mannitol as a bronchial provocation test for airway hyperresponsiveness: a phase 3 comparison study with hypertonic (4.5%) saline. Respiratory Research 2005, 6(144):144. Anderson et al. Comparison of mannitol and methacholine to predict exercise-induced bronchoconstriction and a clinical diagnosis of asthma, Respiratory Research 2009, 10:4

The FEV₁ will be measured 60 seconds after each mannitol dose (0, 5, 10, 20, 40, 80, 160, 160, 160 mg). The subjects will be asked to exhale completely before taking a controlled deep inspiration from the device and to hold their breath for 5 seconds then exhale through their mouth before removal of the nose clip. Sixty seconds after inhalation of the 0 mg capsule the FEV₁ will be measured in duplicate. The highest of these values will be taken as the baseline FEV₁ and will be used to calculate the target FEV₁ value that indicated at least a 15% fall in response to the mannitol challenge. The test result expressed is a PD₁₅.

The procedure outlined above for the 0 mg capsule will be repeated for each dose step until at least a 15% fall in FEV₁ is achieved (or at least a 10% fall between consecutive doses) or the cumulative dose of 635 mg have been administered.

### Exhaled Nitric Oxide

Airway inflammatory and epithelial cells express enzymes called nitric oxide synthases (NOS). These enzymes produce the gas nitric oxide (NO) that diffuses into the airway lumen. Because pulmonary disease processes have varying degrees of inflammation and thus differing amounts of cells containing NOS, levels of NO within the airway lumen will differ as well. Therefore, FE_{NO} increasingly is measured as an indicator of airway inflammation. FE_{NO} varies with expiratory air flow. With this system, a constant exhalation flow must be maintained to achieve a reproducible measure. Silkoff et al. Marked flow dependence of exhaled nitric oxide using a new technique to exclude nasal nitric oxide. Am J Resp Crit Care Med. 155(1): 260-267, 1997 Jan. Recommendations for Standardized Procedures for the Online and Offline Measurement of Exhaled Lower Respiratory Nitric Oxide and Nasal Nitric Oxide in Adults and Children. Am J Resp Crit Care Med. Vol 160:2104-2117, 1999.

Clinical research areas will follow ATS standards for FE_{NO}, or ensure that protocol-specific criteria is met if it is different from ATS standards.

NIOX® Nitric Oxide system, NIOX® filter and NO calibration gas will be obtained from Aerocrine, Inc. Calibration is to be performed every 14 days and/or when the system prompts to calibrate.

Before testing, subjects should refrain from performing exercise and other pulmonary function testing for at least 60 minutes prior to FE_{NO} measurements. Subjects should rinse the mouth prior to testing and should refrain from eating or drinking anything (e.g., caffeine, green vegetables) 30 minutes prior to testing. NIOX® Nitric Oxide system will be used according to NIOX® Daily Use Manual.

### EXAMPLE 3

12 patients with asthma will be treated with oseltamivir phosphate (Tamiflu®) at suitable dosage, e.g., the prescribing information (PI) dosage. The patients may be assigned randomly to several groups: high dose group will receive oseltamivir 150 mg twice daily or equivalent dose adjusted for age, weight, and kidney function for 5 to 10 days; intermediate dose will receive oseltamivir 110 mg twice daily or equivalent dose adjusted for age, weight, and kidney function for 5 to 10 days; standard dose will receive oseltamivir 75 mg twice daily or equivalent dose adjusted for age, weight, and kidney function for 5 to 10 days. http://clinicaltrials.gov/ct2/show/NCT00298233.

Pulmonary function tests will be conducted before administration and at different time points after the administration of Tamiflu® to measure, for example, FEV₁, FEV₁/FVC ratio, PEFR, airway resistance and airway hyperresponsiveness. Asthma-specific quality-of-life scores will be studied using suitable questionnaires (as described more fully in Detailed Description of the Invention and in Example 4). The study will also include a placebo control group although it will not be double blinded. The number of patients might be increased and the selection of patients can be determined based upon their level of asthmatic disease and current level of disease control. None of these patients will exhibit symptoms of a viral syndrome such as fever, myalgias, cough, constitutional symptoms, etc.

Whether the co-administration of antiviral agents could allow lower dosage of anti-inflammatory agents, long-acting β₂-agonists, or short-acting β₂-agonists will also be contemplated for this study or in a future study.

### EXAMPLE 4

### Effects of Antiviral Agents on Bronchial Epithelial Cells from Asthmatic Patients

The expression levels of some genes in bronchial epithelial cells from asthmatics are up- or down-regulated in comparison with cells from healthy individuals. For example, chloride channel, calcium-activated, family member 1 (CLCA1), periostin, serpinB2 and granulocyte macrophage-colony-stimulating factor (GM-CSF) were up-regulated in asthma. Woodruff et al., Proceedings of the National Academy of Sciences USA, 104 (40): 15858-158 (2007). Vachier et al., Am. J. Respir. Crit. Care Med., 158, 3: 963-970 (1998).

To explore epithelial cell dysfunction in asthma and the therapeutic effects of antiviral agents, bronchial epithelial cells will be collected using bronchoscopy from asthmatic or healthy subjects before and at several time points after the administration of antiviral agents. In particular, a double-blind randomized controlled trial will be conducted in which oseltamivir phosphate 98.5 mg (equivalent to oseltamivir 75 mg) or a placebo is administered to asthmatic or healthy subjects twice a day. Alternatively, about 75 mg oseltamivir will be administered to subjects once per day. Bronchoscopy will be performed at baseline and repeated 1 week, 2 weeks, 3 weeks and 4 weeks after starting study. Total mRNA and protein will be prepared from the bronchial epithelial cells. mRNA levels will be studied using RT-PCR or cDNA microarray; protein levels will be studied by ELISA, immunohistochemistry, Western blot or protein microarray. mRNA (or protein) levels in bronchial epithelial cells from asthmatic subjects before, during and after treatment with either Tamiflu® or placebo will be compared.

For gene expression microarray analysis, 25 ng of total epithelial RNA will be amplified by using NuGen Ovation RNA amplification system. Then, 2.75 μg of single-stranded cDNA will be hybridized to Affymetrix (Santa Clara, Calif.) U133 plus 2.0 arrays (>54,000 probe sets coding for 38,500 genes). Array images will be analyzed by using Affymetrix GeneChip Expression Analysis Software. Bioconductor will be used for quality control (affyPLM algorithm), preprocessing (RMA algorithm), cluster analysis, and linear modeling.

Alternatively, primary bronchial epithelial cells will be grown from bronchial brushings (>95% epithelial cells) obtained from normal or asthmatic subjects by fiber-optic bronchoscopy in accordance with standard guidelines. Hurd, et al., Workshop summary and guidelines: investigative use of bronchoscopy. J. Allergy Clin. Immunol. 88:808-814 (1991). Primary cultures will be established by seeding freshly brushed bronchial epithelial cells into hormonally supplemented bronchial epithelial growth medium (Clonetics) containing 50 U/ml penicillin and 50 μg/ml streptomycin. Wark, et al. Asthmatic bronchial epithelial cells have a deficient innate immune response to infection with rhinovirus. Journal of Experimental Medicine, 201 (6): 937-947 (2005). Cultured bronchial epithelial cells will be treated with different doses of oseltamivir phosphate. The levels of mRNA or protein will be studied at different time points after treatment with oseltamivir phosphate.

### EXAMPLE 5

### Effects of Regularly-Scheduled and As-Needed Antiviral Agent Administration in Patients with Asthma

Patients will be randomly assigned to one of two treatment groups and be treated for 16 weeks. Treatment group A will take Tamiflu® on a regular basis (oseltamivir phosphate 98.5 mg (equivalent to oseltamivir 75 mg) twice a day or about 75 mg oseltamivir once per day) plus Tamiflu® as needed. Treatment group B will take placebo on a regular basis (twice per day) plus Tamiflu® as needed. Patients are to record the time and dosage of Tamiflu® taken each day. Patients will be instructed to record their PEFR twice daily using a Mini-Wright peak flow meter (Clement Clarke, Columbus, Ohio) and asthma symptoms once daily in a diary. Patients will take the first capsule in the morning after recording their morning PEFR, and on retiring to sleep after recording their evening PEFR.

While patients receive blinded treatment, the asthma condition will be monitored daily, through PEFRs and symptoms recorded by patients, the change in FEV₁ before and after taking the medication, the concentration of methacholine required to decrease the FEV₁ by 20 percent (PC₂₀), asthma exacerbations, and treatment failure.

Clinic visits by the physicians will be scheduled every two to three weeks. Patients will refrain from taking their study medications for at least eight hours before all clinic visits. During clinic visits, to determine the spirometric response to a medication (Collins Eagle 2 spirometer, Quincy, Mass.), the difference in the FEV₁ before and 30 minutes after administration of studied medication will be measured and reported as percent improvement. To measure PC₂₀ for methacholine, methacholine aerosols will be generated with a nebulizer (model 646, DeVilbiss Health Care, Somerset, Pa.) and a calibrated dosimeter (S&M Instruments, Dovestown, Pa.). The PC₂₀ for methacholine will be determined by standard procedures (Tashkin et al., Am. Rev. Respir. Dis. 145:301, 1992). Asthma exacerbations will be monitored during each clinic visit. Patients will be asked about their asthma control, and all asthma exacerbations will be recorded.

Asthma-specific quality-of-life scores will be recorded during clinic visits, using Asthma Quality of Life Questionnaire (AQLQ) and Living with Asthma Questionnaire (LWAQ). Juniper et al., Development of a Questionnaire for Use in Clinical-Trials. Thorax 47:76-83, 1992. Hyland et al., A Scale for Assessing Quality-of-Life in Adult Asthma Sufferers. Journal of Psychosomatic Research 1991; 35(1):99-110.

FEV₁/forced vital capacity (FVC) ratio, airway resistance, airway hyperresponsiveness (AHR) may also be monitored for this study. Harrison's Principles of Internal Medicine, 17^{th} edition, published by McGraw-Hill Companies, Inc. 2008.

At the completion of the randomized 16-week treatment period, all patients will be switched to single-blind treatment with placebo for a 4-week withdrawal period.

During this time patients will continue to use Tamiflu® as needed. U.S. Pat. No. 6,156,503.

### EXAMPLE 6

### Treatment of Exercise-Induced Asthma

Four to 28 days prior to the start of this study, screening procedures will be performed with male patients 18 and 45 years of age with a history of exercise-induced asthma and a history of at least one year of mild stable asthma (FEV₁≧70% predicted normal value) who required only short-acting β₂-agonists for routine asthma control. One screening and one baseline exercise-challenge test will take place during the 28-day pre-randomization phase. After screening, fifteen patients will be administrated with oseltamivir phosphate 98.5 mg (equivalent to oseltamivir 75 mg) twice a day or about 75 mg oseltamivir once per day, respectively, for 29 days. Fifteen patients will receive placebo for 29 days.

The efficacy of Tamiflu® will be examined in improving (i.e., reducing) the percentage fall in FEV₁ following exercise-challenge testing in subjects with mild asthma. The primary efficacy variable will be maximum post exercise percentage fall index (% FI) at Day 29 and will be calculated as follows:

$$\% F I = \frac{preexercise F E {V}_{1} - lowest F E {V}_{1} ( after excercise )}{preexercise F E {V}_{1}} \times 100$$

The pharmacodynamic studies will include absolute change in resting AM online measurements of exhaled nitric oxide on Days 1, 15, and 29; ex vivo LPS-stimulated TNF-α levels measurements in whole blood on Days 1 and 29; and whole blood gene expression (mRNA) analysis of inflammatory mediators on Days 1 and 29.

For pharmacokinetic study, area under the concentration versus time curve (AUC), maximum concentration (Cmax), terminal elimination half-life (T_{1/2}), time to maximum concentration (Tmax), apparent clearance (CL/F), volume of distribution (V_{d}), and relative accumulation rate (RA) will be measured.

Safety study will include observations of adverse events; clinical laboratory evaluations, e.g., urinalysis, chemistry, hematology including absolute white blood cell counts and anti-nuclear antibody; physical examination findings; vital sign measurements; 12-lead electrocardiogram recordings; and global assessment of atopic dermatitis (for subjects with atopic dermatitis at screening). U.S. Pat. No. 7,276,529.

### EXAMPLE 7

### Experiment with a Murine Model of House Dust Mite Extract (HDM)-Induced Asthma

Normal BALB/c mice that are 6-8 weeks old will be exposed to 25 μg of commercially-prepared purified HDM extract or saline (control mice) intranasally for 5 days per week for up to 10 weeks (Johnson et al., 2004). During this period, the HDM-exposed animals will display the entire disease progression, from mild asthma to severe asthma, with airway hyper-responsiveness beginning at the 5th week. To evaluate asthma progression, several tests will be performed, including eosinophil counts in bronchoalveolar lavage fluid and serum cytokine measurements as described in Evans et al., (2003) J Appl Physiol 94:245-52. Cell counts and differential staining will be performed, and cytokine levels of IL-4, IL-13, IL-5 and TNF-α will be performed at 2-week intervals after exposure to HDM. Total IgE and HDM-specific IgE levels in the serum will be measured using antigen-capture ELISA. Respiratory function will be assessed by measurements of airway resistance and lung compliance using the methacholine dose-response method. Glaab et al., (2005) Respir Res 6:139-48. Airway hyper-responsiveness to methacholine challenge is a well-characterized measure of respiratory function, especially the assessment of airway resistance.

Airway morphometric measurements will be used to assess the thickness of bronchial walls using standard methods. Kuhn et al., (2000) Am J Respir Cell Mol Biol 22:289-295. Bronchial lumen diameter, epithelial thickness, and the bronchial wall thickness will be measured.

Protein expression in animals made asthmatic by the above procedure will be compared to control untreated animals. Lung and trachea samples will be obtained from test and control animals at baseline (before HDM exposure), after 1 week, 3 weeks, 5 weeks, 7 weeks, and 10 weeks. Animals will be euthanized by carbon dioxide overdose followed by cervical dislocation, followed by immediate tissue sampling.

Immunohistochemistry (IHC) to analyze gene expression will be performed on tissue samples as described in Mason et al., (2004) Endocrinology 145 (2): 976-982. Two portions of tissue from each site will be frozen at −80° C. for preparation of mRNA and tissue protein lysate preparation. Fresh samples of trachea will be used to prepare cultured airway smooth muscle cells (ASM) as described below. Histological analysis of HDM ASM will be carried out using hematoxylin and eosin, trichrome, and Sirius red using standard protocols and examined for gross histological changes in the trachea and lung during the course of asthma development in the HDM stimulated mouse, and comparisons will be made between baseline (before HDM exposure), and after 1 week, 3 weeks, 5 weeks, and 7 weeks.

CCN5 is greatly reduced in smooth muscle cells in the airways of asthmatic mouse in comparison to control mouse. In order to study the effect of antiviral agents on treating asthma, the expression level of CCN5 will be analyzed before, during and after the administration of antiviral agents to asthmatic mouse. The expression level of other proteins which are up- or down-regulated in asthmatic mouse may also be analyzed using the method described.

CCN5 protein analysis by IHC of normal and HDM ASM will be performed using each of frozen and paraffin sections of lung and trachea and antibodies for CCN5. CCN5 expression patterns will be examined in normal lung and trachea and compared to that of tissues obtained at baseline (before HDM exposure), and after 1 week, 3 weeks, 5 weeks, and 7 weeks. Slides will be stained for CCN5 expression using the DAB-peroxidase. The lack of CCN5 expression in the smooth muscle cells could thus be causally associated with the disease state.

The airway responses to administered Tamiflu® will be analyzed in mice exposed to HDM extract using the methods described above. Animals will be monitored for 10 weeks from the beginning of Tamiflu® administration. Respiratory function tests will be based on measurements of airway dose-response curves to intravenous methacholine as described above. These tests will be performed every two weeks following the first administration of Tamiflu®. Airway morphometric measurements to determine bronchial wall thickness will be performed as described above. At two week intervals, trachea and lung samples will be isolated and analyzed histologically and by IHC for the expression of CCN5, CCN2, smooth muscle α-actin, and matrix metalloproteinases and inhibitors: MMP-9, MMP-8, MMP-2, MMP-12, MT1-MMP, and TIMPs 1 and 2.

Tracheal and bronchial samples will be isolated for preparation of cultured murine

ASM for use in the examples described below, for analysis of ASM at all stages of asthma. Tracheal and bronchial samples will be excised and transferred immediately to ice-cold buffer. ASM will be isolated and cultured using a standard method. Deshpande et al., (2005) Am J Resp Cell Molec Biol 32:149-156. The smooth muscle phenotype will be confirmed by IHC staining using antibodies to smooth muscle-specific α-actin and smooth muscle myosin heavy chain.

ASM will be tested for CCN5 protein and mRNA levels in ASMC obtained from each of the different HDM time points discussed above. Protein levels will be analyzed by Western blot. mRNA levels also will be analyzed by Q-PCR. The cDNA prepared from ASM cells will be analyzed by microarray for gene expression profiling.

To test whether antiviral agents have a role in regulation of ASM function in vivo and are effective in the treatment of asthma, Tamiflu® will be administered to HDM-exposed mice and prevention and arrest of ASM hyper-proliferation and airway remodeling (AWR) will be analyzed.

Tamiflu® will be administered to the mice from three weeks after the beginning of HDM exposure (after the onset of the first phase of asthma) to five weeks after HDM (after the onset of ASM hyper-proliferation). The following groups of animals will be analyzed: (1) four animals exposed to intranasal HDM for an entire 5 week test period and to Tamiflu® for weeks 3-5; (2) four animals exposed to HDM for all 5 weeks and a placebo for weeks 3-5; (3) two control animals exposed to HDM for all 5 weeks only; and (4) two control animals exposed to a placebo only for weeks 3-5. Animals in groups 1-3 above will be administered HDM for 5 days each week, and Tamiflu® will be administered twice during weeks 3-5 (groups 1, 2, and 4).

At the end of the five-week HDM exposure period, animals will be tested for respiratory function to estimate airway dose-response after intravenous methacholine (MCh) administration. After the tests of airway function, animals will be sacrificed by thoracotomy to examine airway histologies. Trachea and lung samples will be isolated and fixed, and paraffin sections are analyzed by conventional histology and by immunohistochemistry for the expression of CCN5. Blinded results of airway function testing and airway morphology will be analyzed. U.S. Patent Publication No. 20080207489.

### EXAMPLE 8

### Experiment with a Murine Model of Ovalbumin-Induced Asthma

Activities of Tamiflu® will be investigated in a murine model of ovalbumin (OVA)-induced asthma. The effects on airway hyperresponsiveness (AHR), cellular infiltrate into the BAL and tissue, plasma total IgE levels, whole lung cytokine levels, and goblet cell hyperplasia in OVA-sensitized mice will be assessed in this study.

Male BALB/c mice will be sensitized to ovalbumin (OVA) via subcutaneous, intraperitoneal, intranasal and intratracheal routes. Animals will be dosed with vehicle (0.5% carboxymethyl-cellulose (CMC)/0.25% Tween 80; p.o.), Budesonide (10 mg/kg; p.o.) or Tamiflu® (1, 10 or 25 mg/kg; q.d.; p.o.) prior to intratracheal challenge with OVA until the end of the study.

Airway hyper-responsiveness (AHR) to methacholine (MCh, 3 mg/kg; i.v.), bronchoalveolar lavage total white blood cell and differential counts, total plasma IgE, whole lung eotaxin, RANTES and IL-4 protein levels, and goblet cell hyperplasia will be assessed 24, 48 and 72 hours after the second intratracheal OVA challenge. The time course of total IgE in the plasma will be measured by ELISA. RANTES, IL-4 and eotaxin will be measured by ELISA in whole lung homogenates. PAS (Periodic Acid Schiff) staining will be used to detect mucosubstances produced by goblet cells within the airways. PAS staining will be carried out on 5 μM sections according to the manufacturers instructions using the PAS staining kit (Polysciences, Pa.). Twenty airways per section will be counted and the PAS positive airways will be expressed as a % of the total airways counted. U.S. Pat. No. 7,276,529.

### EXAMPLE 9

### Effects of Antiviral Agents on Acute Experimental Asthma Induced in Dogs by Methacholine

In one of asthma animal models, bronchospasms can be experimentally induced by inhalation of methacholine in dogs.

To carry out these tests, healthy mongrel dogs weighing 19 to 22 kg will be anesthetized with sodium pentobarbital, 30 mg/kg IV, and intubated with endotracheal tubes. Initially, the ventilation will be maintained with a Harvard ventilator, which serves also to deliver methacholine, with or without aerosolized Tamiflu® solution. Subsequently, the animals will be continuously ventilated prior to and following drug administration. Arterial venous and pulmonary artery (Swan-Ganz) will be inserted, and used to obtain blood samples, monitor vascular pressures and measure cardiac output using thermodilution method. Arterial end pulmonary pressure, electrocardiogram, will constantly be monitored on a DR-8 multichannel recorder. Arterial blood, hemoglobin, pH, PO₂ , PCO₂, and potassium will be measured with Radiometer ABL-4 (Copenhagen).

Upon completion of the surgical preparation, the dogs will be allowed 30 min to stabilize hemodynamically. Several arterial blood samples will be obtained and ventilation will be adjusted so that arterial blood gases and pH are within physiologic range.

The bronchial challenge procedure will entail a 90 sec administration of aerosolized 0.5% methacholine hydrochloride (Sigma). In treating animals, aerosolized Tamiflu® will be administered for 3 minutes via Harvard respirator and microembulizer. In addition, five dogs will be treated with Tamiflu® after (but not before) the administration of methylcholine.

Blood gas measurements will be made at 5, 15, and 30 minutes after drug inhalation is completed. Bronchoprovocation will be either with methacholine alone (n=7), or following pretreatment with Tamiflu® (n=7).

Differences between Tamiflu®-treated and control dogs will be compared by the two-tailed student's unpaired t-test, and probability levels lower than 5% will be considered to be significant. U.S. Pat. No. 5,858,985.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A method of treating asthma in a patient comprising administering to the patient a therapeutically effective amount of an antiviral agent.

2. The method of claim 1, wherein the antiviral agent is administered to the patient by inhalation, nasally, intravenously, orally, subcutaneously, intramuscularly or transdermally.

3. The method of claim 1, wherein the antiviral agent is formulated for delivery as aerosols to the patient.

4. The method of claim 1 wherein there is at least about a 10% increase in forced expiratory volume in 1 second (FEV₁) within about 30 minutes to about 14 days after administration of the antiviral agent.

5. The method of claim 4 wherein there is at least about a 10% increase in FEV₁ within about 2 hours to about 12 days after administration of the antiviral agent.

6. The method of claim 5 wherein there is at least about a 10% increase in FEV₁ within about 1 day to about 11 days after administration of the antiviral agent.

7. The method of claim 6 wherein there is at least about a 10% increase in FEV₁ within about 2 days to about 10 days after administration of the antiviral agent.

8. The method of claim 4 wherein there is at least about a 20% increase in FEV₁ within about 30 minutes to about 14 days after administration of the antiviral agent.

9. The method of claim 8 wherein there is at least about a 20% increase in FEV₁ within about 2 hours to about 12 days after administration of the antiviral agent.

10. The method of claim 9 wherein there is at least about a 20% increase in FEV₁ within about 1 day to about 11 days after administration of the antiviral agent.

11. The method of claim 10 wherein there is at least about a 20% increase in FEV₁ within about 2 days to about 10 days after administration of the antiviral agent.

12. The method of claim 8 wherein there is at least about a 30% increase in FEV₁ within about 30 minutes to about 14 days after administration of the antiviral agent.

13. The method of claim 12 wherein there is at least about a 30% increase in FEV₁ within about 2 hours to about 12 days after administration of the antiviral agent.

14. The method of claim 13 wherein there is at least about a 30% increase in FEV₁ within about 1 day to about 11 days after administration of the antiviral agent.

15. The method of claim 14 wherein there is at least about a 30% increase in FEV₁ within about 2 days to about 10 days after administration of the antiviral agent.

16. The method of claim 1 wherein there is at least about a 10% increase in peak expiratory flow rate within about 30 minutes to about 14 days after administration of the antiviral agent.

17. The method of claim 16 wherein there is at least about a 10% increase in peak expiratory flow rate within about 2 hours to about 12 days after administration of the antiviral agent.

18. The method of claim 17 wherein there is at least about a 10% increase in peak expiratory flow rate within about 1 day to about 11 days after administration of the antiviral agent.

19. The method of claim 18 wherein there is at least about a 10% increase in peak expiratory flow rate within about 2 days to about 10 days after administration of the antiviral agent.

20. The method of claim 16 wherein there is at least about a 20% increase in peak expiratory flow rate within about 30 minutes to about 14 days after administration of the antiviral agent.

21. The method of claim 20 wherein there is at least about a 20% increase in peak expiratory flow rate within about 2 hours to about 12 days after administration of the antiviral agent.

22. The method of claim 21 wherein there is at least about a 20% increase in peak expiratory flow rate within about 1 day to about 11 days after administration of the antiviral agent.

23. The method of claim 22 wherein there is at least about a 20% increase in peak expiratory flow rate within about 2 days to about 10 days after administration of the antiviral agent.

24. The method of claim 16 wherein there is at least about a 30% increase in peak expiratory flow rate within about 30 minutes to about 14 days after administration of the antiviral agent.

25. The method of claim 24 wherein there is at least about a 30% increase in peak expiratory flow rate within about 2 hours to about 12 days after administration of the antiviral agent.

26. The method of claim 25 wherein there is at least about a 30% increase in peak expiratory flow rate within about 1 day to about 11 days after administration of the antiviral agent.

27. The method of claim 26 wherein there is at least about a 30% increase in peak expiratory flow rate within about 2 days to about 12 days after administration of the antiviral agent.

28. The method of claim 1, wherein the antiviral agent is a neuraminidase inhibitor.

29. The method of claim 28, wherein the neuraminidase inhibitor is selected from the group consisting of oseltamivir, zanamivir and peramivir.

30. The method of claim 1, wherein the antiviral agent is a viral fusion inhibitor.

31. The method of claim 1, wherein the antiviral agent is a protease inhibitor.

32. The method of claim 1, wherein the antiviral agent is a DNA polymerase inhibitor.

33. The method of claim 1, wherein the antiviral agent is a signal transduction inhibitor.

34. The method of claim 1, wherein the antiviral agent is a nucleoside reverse transcriptase inhibitor (NRTI).

35. The method of claim 1, wherein the antiviral agent is a non-nucleoside reverse transcriptase inhibitor (NNRTI).

36. The method of claim 1, wherein the antiviral agent is an interferon.

37. A pharmaceutical composition for treatment of asthma in a patient comprising a therapeutically effective amount of an antiviral agent.

38. The pharmaceutical composition of claim 37 formulated for delivery of the antiviral agent as aerosols to the patient.

39. The pharmaceutical composition of claim 37, wherein the pharmaceutical composition is administered by inhalation, nasally, intravenously, orally, subcutaneously, intramuscularly or transdermally.

40. The pharmaceutical composition of claim 37 wherein the antiviral agent is selected from the group consisting of a neuraminidase inhibitor, a viral fusion inhibitor, a protease inhibitor, a DNA polymerase inhibitor, a signal transduction inhibitor, a reverse transcriptase inhibitor and an interferon.

41. An article of manufacture comprising a pharmaceutical formulation of an antiviral agent for treatment of asthma and printed matter indicating that the pharmaceutical formulation should be inhaled by a patient suffering from asthma.

42. The article of manufacture of claim 41 wherein the antiviral agent is selected from the group consisting of a neuraminidase inhibitor, a viral fusion inhibitor, a protease inhibitor, a DNA polymerase inhibitor, a signal transduction inhibitor, a reverse transcriptase inhibitor and an interferon.
